(19) United States
(12) Patent Application Publication
(10) Pub. No.: US 2023/0346807 A1
(43) Pub. Date: Nov. 2, 2023

(54) METHODS FOR PRODUCING CANNABINOID-CONTAINING CRYSTALS USING SUPERCRITICAL FLUID

(71) Applicant: CALDERA GROUP LLC, MEDFORD, OR (US)

(72) Inventors: CHASE KENYON MCMICHAEL, AUSTIN, TX (US); JACK ANTHONY GRAHAM GALPIN, MEDFORD, OR (US)

(21) Appl. No.: 17/793,277

(22) PCT Filed: Jan. 18, 2021
(86) PCT No.: PCT/US21/13812

§ 371 Date: Jul. 15, 2022

Related U.S. Application Data
(60) Provisional application No. 62/961,803, filed on Jan. 16, 2020.

(51) Int. Cl. A61K 31/00; A61K 36/185; B01D 11/02; B01D 5/00

(52) U.S. Cl. CPC A61K 31/658; A61K 36/185; B01D 11/0203; B01D 5/0036; B01D 5/009; A61K 2236/37; A61K 2236/53; A61K 2236/31

(57) ABSTRACT

A method for producing cannabinoid-containing crystals includes providing a CBGA-containing plant material that includes at least 2% CBGA by dry weight. The CBGA-containing plant material is exposed to a fluid in a supercritical state to extract components of the CBGA-containing plant material. A fraction of the extracted components is coalesced. The coalesced fraction of the extracted components includes cannabinoid-containing crystals containing at least one of CBGA and a cannabinoid derivative of CBGA from the carbon dioxide.

### PRIORITY

The present application claims the priority of United States Provisional Patent Application No. 62/961,803, filed Jan. 16, 2020, which is incorporated herein by reference in its entirety.

### FIELD

The present application relates to the field of extraction, chemical conversion, separation, and crystallization of cannabigerolic acid (CBGA) and derivatives thereof.

### BACKGROUND

Cannabinoids are valuable plant-derived natural products. CBGA is a valuable central branch-point intermediate for the biosynthesis of the different major classes of cannabinoids. Alternative cyclization of the prenyl side-chain of CBGA yields tetrahydrocannabinolic acid (THCA) or its isomers cannabidiolic acid (CBDA) or cannabichromenic acid (CBCA). Leading-edge work identified and purified three enzymes responsible for these cyclizations. The genes for THCA synthase and CBDA synthase have been reported in Japan.

The mechanism of how CBGA is created within the cannabis plant was discovered relatively recently. The first enzymatic step in CBGA biosynthesis is the formation of olivetolic acid by a putative polyketide synthase enzyme, termed olivetolic acid synthase. The second enzymatic step in CBGA biosynthesis is the prenylation of olivetolic acid to form cannabigerolic acid (CBGA) by the enzyme geranylpyrophosphate:olivetolate geranyltransferase. Using crude protein extracts of cannabis leaves, the enzyme has been identified that catalyzed the prenylation of olivetolic acid with geranyl diphosphate.

Genes encoding enzymes of cannabinoid biosynthesis will be useful in metabolic engineering of cannabis varieties that contain ultra-low levels of THC and other cannabinoids. Cross-hybridization of distinct homozygous cannabis plants to produce consistent early flowering seeds create such material to produce high cannabigerolic acid plant material. Such genes may also prove useful for the creation of specific cannabis varieties for the production of cannabinoid-based pharmaceuticals, or for reconstituting cannabinoid biosynthesis in other organisms such as bacteria or yeast. The enzyme identification and further creation of metabolically engineering cannabis established a foundation for extraction and further refinement.

In recent years, mechanical extraction, solvent extraction, and extractions, supported by different physical methods and means, have been used in the field of preparation of various plant extracts. Supercritical CO₂ extraction (SCCO₂E) has been used for the extraction of fragrances and essential oils from natural materials. The SCCO₂E is a separation technology that uses the supercritical fluid as the solvent. Above the critical temperature, fluids cannot be liquefied regardless of the pressure applied however they can reach a density close to the liquid state.

A technique of formulation of crystals using SCCO2 is well-published whereby supercritical solutions expand across a nozzle leveraging a rapid pressure drop inside the nozzle, which initiates precipitation of the solute from the solution driven by nucleation, condensation and particle coagulation. These techniques necessitate difficult subsequent separation steps and do not enable sufficient control of the resulting cannabinoids.

Accordingly, those skilled in the art continue with research and development in the field of extraction, chemical conversion, separation, and crystallization of CBGA and derivatives thereof.

### SUMMARY

In one embodiment, a method for producing cannabinoid-containing crystals includes providing a CBGA-containing plant material that includes at least 2% CBGA by dry weight. The CBGA-containing plant material is exposed to a fluid in a supercritical state to extract components of the CBGA-containing plant material. A fraction of the extracted components is coalesced. The coalesced fraction of the extracted components includes cannabinoid-containing crystals containing at least one of CBGA and a cannabinoid derivative of CBGA from the carbon dioxide.

In another embodiment, a method for producing cannabinoid-containing crystals includes placing CBGA-containing plant material that includes at least 2% CBGA by dry weight into an extractor. Carbon dioxide in a subcritical liquid state is provided to the extractor containing the CBGA-containing plant material. The carbon dioxide within the extractor is transformed from the subcritical state to a supercritical state to extract components from the CBGA-containing plant material. The carbon dioxide containing the extracted components of the CBGA-containing plant material is flowed to a first separator. A pressure of the carbon dioxide flowed into the first separator is decreased to coalesce a fraction of the extracted components from the carbon dioxide. The coalesced fraction of the extracted components includes cannabinoid-containing crystals containing at least one of CBGA and a cannabinoid derivative of CBGA. The carbon dioxide containing an uncoalesced fraction of the extracted components is flowed out of the first separator. The cannabinoid-containing crystals are collected from the first separator.

Other embodiments of the disclosed methods for producing cannabinoid-containing crystals using supercritical fluid will become apparent from the following detailed description, the accompanying drawings and the appended claims.

### BRIEF DESCRIPTION OF THE DRAWINGS

FIG. 1 illustrates an exemplary supercritical fluid extraction system suitable for use with the methods of the present description.

FIG. 2 illustrates a block diagram of an exemplary supporting electronic system for use with the exemplary supercritical carbon dioxide (SCCO2) extraction system of FIG. 1.

FIG. 3 illustrates a non-exhaustive list of CBGA and cannabinoid derivatives of CBGA.

FIG. 4 illustrates a flow diagram of an exemplary method for producing cannabinoid-containing crystals according to an aspect of the present description.

FIG. 5 illustrates another flow diagram of an exemplary method for producing cannabinoid-containing crystals according to another aspect of the present description.

FIG. 6 shows a CBGA isolate according to the present description as measured on high-performance liquid chromatography.

FIG. 7 is a bar graph showing results of a first experimental example of the present description.

FIG. 8 is a bar graph showing results of a second experimental example of the present description.

FIG. 9 is a bar graph showing results of a third experimental example of the present description.

FIG. 10 is a bar graph showing results of a fourth experimental example of the present description.

### DETAILED DESCRIPTION

The foregoing general description and the brief description of the drawings and the following detailed description are exemplary and explanatory and are intended to provide further explanation of the invention as claimed. Other objects, advantages, and novel features will be readily apparent to those skilled in the art from the following detailed description of the invention

The present description relates to methods for extraction, chemical conversion, separation, and/or crystallization of cannabigerolic acid (CBGA) and derivatives thereof using supercritical fluid.

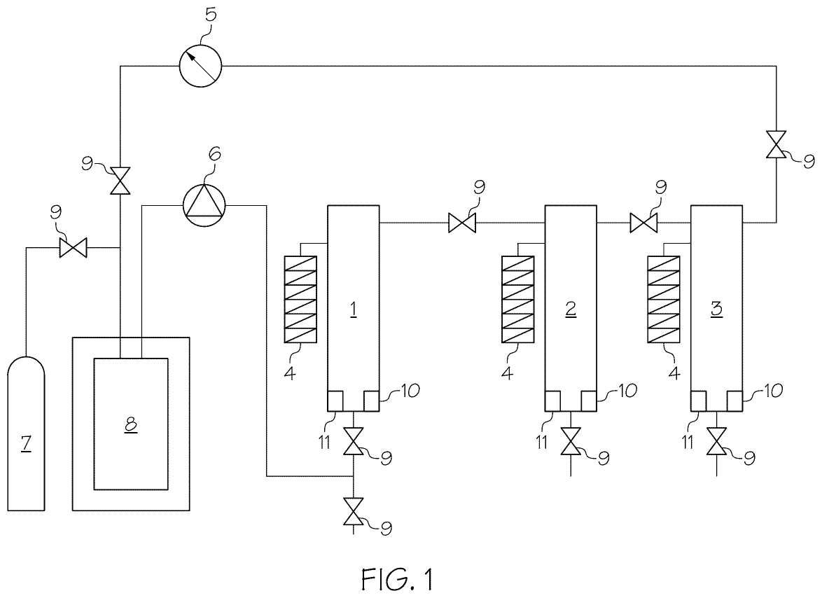

FIG. 1 shows an exemplary supercritical fluid extraction system suitable for use with the methods of the present description. The methods of the present description are not limited to use with the illustrated supercritical fluid extraction system. The methods of the present description may be used with any system suitable for supercritical fluid extraction, chemical conversion, separation, and/or crystallization.

As shown in FIG. 1, the exemplary supercritical fluid extraction system is in the form of a supercritical carbon dioxide (SCCO2) extraction system. In the detailed description below, carbon dioxide is employed at the supercritical fluid. However, the present description includes alternative supercritical fluid extraction systems having supercritical fluids other than carbon dioxide that may be used instead of or in addition to carbon dioxide.

As shown in FIG. 1, the exemplary supercritical carbon dioxide (SCCO2) extraction system includes: an extractor (1) for extracting components of a starting plant material placed in the extractor (1) using carbon dioxide; a first separator (2) for collecting a heavy fraction of components of the starting plant material extracted by the extractor (1); a second separator (3) for collecting a light fraction of components of the starting plant material extracted by the extractor (1); one or more heat exchangers (4) for heating and cooling the extractor (1), the first separator (2), and the second separator (3); a rotameter (5) for measuring a flow rate of the carbon dioxide through the supercritical carbon dioxide (SCCO2) extraction system; a high pressure pump and coriolis meter (6) for flow carbon dioxide and carbon dioxide density monitoring into the extractor (1) under high pressures; carbon dioxide supply components for supplying liquid carbon dioxide to the supercritical carbon dioxide (SCCO2) extraction system, including cylinder (7) for supplying carbon dioxide and conditioning tank (8) to maintain levels and temperature of liquid carbon dioxide; valves (9) for controlling flows of fluids throughout the supercritical carbon dioxide (SCCO2) extraction system; temperature sensors (10) for sensing temperatures within the extractor (1), the first separator (2), and the second separator (3); and pressure sensors (11) for sensing pressures within the extractor (1), the first separator (2), and the second separator (3).

The illustrated supercritical fluid extraction system is exemplary and various variations are included in the scope of the present description. In an alternative example, the extraction function could be omitted, and the starting material could be CBGA and/or derivatives thereof. In another alternative example, the extractor (1) could be used for both extracting components of a starting plant material and then for collecting extracted components of the starting plant material. In alternative supercritical fluid extraction systems, the second separator (3) could be omitted, and the first separator (2) could be used for first collecting a heavy fraction of the components of the starting plant material and then collecting a light fraction of the components of the starting plant material extracted by the extractor (1). In alternative supercritical fluid extraction systems, a third or more additional separators could be added to form a cascade collection at different pressures and temperatures. In alternative supercritical fluid extraction systems, the one or more heat exchangers (4) could be replaced with other heating or cooling devices. In alternative supercritical fluid extraction systems, the rotameter (5) could be replaced with other devices for measuring a flow rate or could be omitted. In alternative supercritical fluid extraction systems, the carbon dioxide supply components (7) and (8) could be replaced with other supply components for supplying carbon dioxide and/or other fluids usable as supercritical fluids.

Returning to the exemplary supercritical carbon dioxide (SCCO2) extraction system of FIG. 1, the extractor (1) includes a pressure vessel capable of withstanding high pressures. The extractor (1) includes an opening (not illustrated) for placing a starting plant material therein at the start of the extraction process and for removing the plant material at the end of the extraction process. The extractor (1) includes an input port for flowing carbon dioxide from carbon dioxide supply components (7) and (8) into the pressure vessel. The extractor (1) include an output port for flowing the carbon dioxide along with extracted components of the starting plant material carried by the carbon dioxide out of the extractor (1) to a first separator (2). The extractor (1) includes a temperature sensor (10) for sensing a temperature of the interior of the pressure vessel. The extractor (1) includes a pressure sensor (11) for sensing a pressure of the interior of the pressure vessel. The extractor (1) includes a heater for heating a temperature of the interior of the pressure vessel. The extractor (1) includes a cooler for cooling a temperature of the interior of the pressure vessel. The heater and cooler are combined in the form of a heat exchanger (4) in fluid communication with the interior of the pressure vessel for controlling a temperature of the interior of the pressure vessel. The extractor (1) includes insulation (e.g. a double-wall jacket) (not illustrated) for maintaining a desired temperature of the interior of the extractor (1). Thus, the extractor (1) is configured to control temperature and pressure conditions for extraction of components of the starting plant material. The described extractor (1) is exemplary and various variations are included in the scope of the present description.

Referring to FIG. 1, the first separator (2) includes a pressure vessel capable of withstanding high pressures. The first separator (2) includes an input port for flowing the carbon dioxide and the extracted components of the starting plant material from the extractor (1) to the pressure vessel of the first separator (2). The first separator (2) includes an opening (not illustrated) for collecting a coalesced heavy fraction of the extracted components of the starting plant material that were carried by the carbon dioxide into the first separator (2) from the extractor (1). The first separator (2) includes an output port for flowing the carbon dioxide along with an uncoalesced light fraction of the extracted components of the starting plant material to the second separator (3). The first separator (2) includes a temperature sensor (10) for sensing a temperature of the interior of the pressure vessel. The first separator (2) includes a pressure sensor (11) for sensing a pressure of the interior of the pressure vessel. The first separator (2) includes a heater for heating a temperature of the interior of the pressure vessel. The first separator (2) includes a cooler for cooling a temperature of the interior of the pressure vessel. The heater and cooler are combined in the form of a heat exchanger (4) in fluid communication with the interior of the pressure vessel for controlling the temperature of the interior of the pressure vessel. The first separator (2) includes insulation (e.g. a double-wall jacket) (not illustrated) for maintaining a desired temperature of the interior of the first separator (2). Thus, the first separator (2) is configured to receive components of the starting plant material extracted in the extractor (1), to coalesce a fraction of the components of the starting plant material at different temperatures and pressures, and to collect the coalesced components. The first separator (2) may also function as a conversion chamber for altering the components of the starting plant material. The described first separator (2) is exemplary and various variations are included in the scope of the present description.

Referring to FIG. 1, the second separator (3) includes a pressure vessel capable of withstanding high pressures. The second separator (3) includes an input port for flowing the carbon dioxide and the light fraction of the extracted components of the starting plant material from the first separator (2). The second separator (3) includes an opening (not illustrated) for collecting a coalesced light fraction of the extracted components of the starting plant material that were carried by the carbon dioxide into the second separator (3) from the first separator (2). The second separator (3) includes an output port for flowing the carbon dioxide out of the second separator (3). The second separator (3) includes a temperature sensor (10) for sensing a temperature of the interior of the pressure vessel. The second separator (3) includes a pressure sensor (11) for sensing a pressure of the interior of the pressure vessel. The second separator (3) includes a heater for heating a temperature of the interior of the pressure vessel. The second separator (3) includes a cooler for cooling a temperature of the interior of the pressure vessel. The heater and cooler are combined in the form of a heat exchanger (4) in fluid communication with the interior of the pressure vessel for controlling a temperature of the interior of the pressure vessel. The second separator (3) includes insulation (e.g. a double-wall jacket) (not illustrated) for maintaining a desired temperature of the interior of the second separator (3). Thus, the second separator (3) is configured to receive components of the starting plant material passed through the first separator (2), to coalesce at different temperatures and pressures a fraction of or all remaining components of the starting plant material not extracted by the first separator (2), and to collect the coalesced components. The second separator (3) may also function as a conversion chamber for altering the components of the starting plant material. The described second separator (3) is exemplary and various variations are included in the scope of the present description.

As shown in FIG. 1, the exemplary supercritical carbon dioxide (SCCO2) extraction system further includes various valves (9) for controlling pressures of the extractor (1), the first separator (2), and the second separator (3) and for controlling flow of carbon dioxide throughout the system.

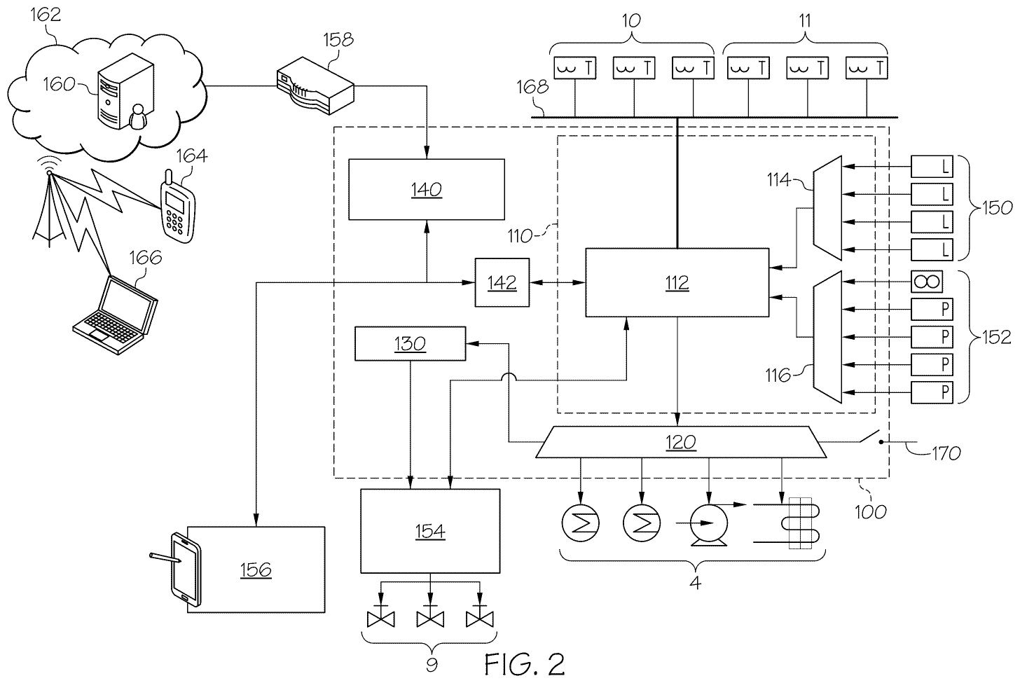

FIG. 2 illustrates a block diagram of an exemplary supporting electronic system for use with the exemplary supercritical carbon dioxide (SCCO2) extraction system of FIG. 1. The exemplary supporting electronic system of FIG. 2 receives temperature and pressure information sensed by the temperature sensors (10) and pressure sensors (11) of the extraction system of FIG. 1, controls the valves (9) throughout the extraction system of FIG. 1, and controls the heaters and coolers (e.g. combined in the form of heat exchangers (4) of the extractor (1), first separator (2), and second separator (3)). The methods of the present description are not limited to use with the illustrated supporting electronic system. The methods of the present description may be used with any control system suitable for controlling the temperature and/or the pressure of the supercritical extraction system. The supporting electronic system facilitates the capabilities of the extraction system to maintain and manage temperature, pressure and flow rate as well as a programming interface to a control total run time based on flow rate (e.g. kg/min) as a function of total starting plant material being processed. The electronic controls of the supporting electronic system facilitate the management of temperature and pressure changes over time.

More specifically, FIG. 2 that the supporting electronic system may include, for example, control system (100) that receives temperature and pressure information sensed by the temperature sensors (10) and pressure sensors (11), receives digital inputs (150) and analogue inputs (152) corresponding to any other various inputs desired for controlling the extraction system, controls the valves (9) by way of a motorized value control system (154), communicates with a user interface (156) associated with the extraction system, communicates with a VPN router (158) that communicates with a VPN server (160) by way of internet (162) that communicates with, for example, a cell phone (164) or personal computer (166), and receives power from an external power source (170) such a AC 230 volt power source. The control system (100) may include, for example, a microcontroller (110), a power control (120), a power supply (130), an ethernet converter (140), and a communication interface (142). The microcontroller (110) receives temperature and pressure information sensed by the temperature sensors (10) and pressure sensors (11). The microcontroller (110) receives digital inputs (150) and analogue inputs (152) corresponding to any other various inputs desired for controlling the extraction system. The microcontroller (110) sends control signals to power control (120) that receives power from external power source (170), which controls valves (9) and sends power to power supply (130). The microcontroller (110) communicates with communication interface (142), which communicates with user interface (156) and to the VPN router (158) by way of ethernet converter (140). The microcontroller (110) may include, for example, central processing unit (112), general-purpose input/output (GPIO) framework (114) receiving digital inputs (150) and analog to digital converter (ADC) interface (116) receiving analog inputs (152).

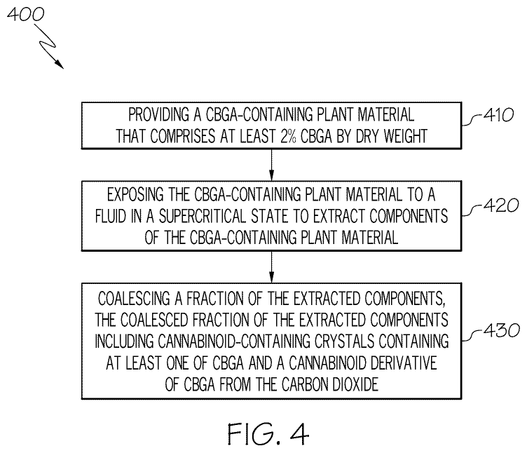

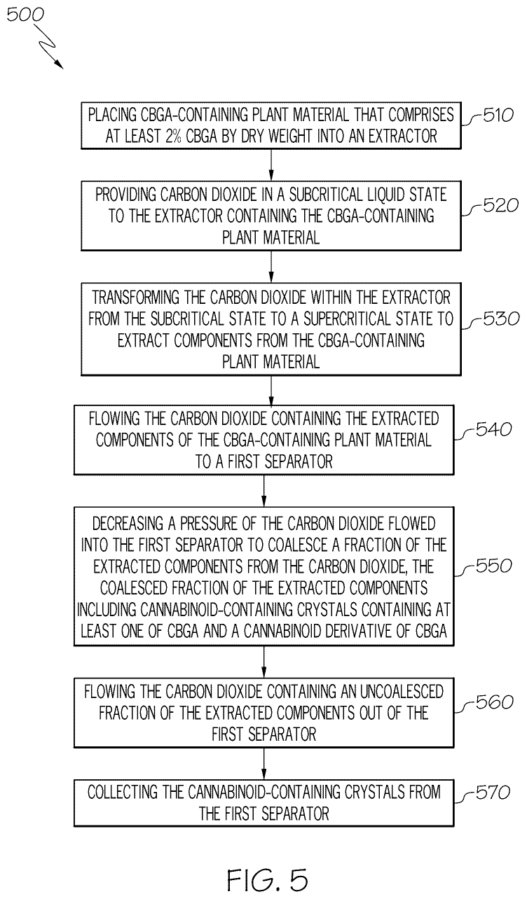

FIGS. 4 and 5 illustrates flow diagrams of exemplary methods for producing cannabinoid-containing crystals according to originally claimed aspects of the present description. As shown in FIG. 4, an exemplary method for producing cannabinoid-containing crystals, at block 400, includes the steps of: at block 410, providing a CBGA-containing plant material that comprises at least 2% CBGA by dry weight; at block 420, exposing the CBGA-containing plant material to a fluid in a supercritical state to extract components of the CBGA-containing plant material; and at block 430, coalescing a fraction of the extracted components, the coalesced fraction of the extracted components including cannabinoid-containing crystals containing at least one of CBGA and a cannabinoid derivative of CBGA from the carbon dioxide. As shown in FIG. 5, an exemplary method for producing cannabinoid-containing crystals, at block 500, includes the steps of: at block 510, placing CBGA-containing plant material that comprises at least 2% CBGA by dry weight into an extractor; at block 520, providing carbon dioxide in a subcritical liquid state to the extractor containing the CBGA-containing plant material; at block 530, transforming the carbon dioxide within the extractor from the subcritical state to a supercritical state to extract components from the CBGA-containing plant material; at block 540, flowing the carbon dioxide containing the extracted components of the CBGA-containing plant material to a first separator; at block 550, decreasing a pressure of the carbon dioxide flowed into the first separator to coalesce a fraction of the extracted components from the carbon dioxide, the coalesced fraction of the extracted components including cannabinoid-containing crystals containing at least one of CBGA and a cannabinoid derivative of CBGA; at block 560, flowing the carbon dioxide containing an uncoalesced fraction of the extracted components out of the first separator; and at block 570, collecting the cannabinoid-containing crystals from the first separator.

The methods of the present description may include a step of preparing a CBGA-containing plant material for extraction. Note that the methods of the present description do not necessarily include extraction. Thus, the methods of the present description may relate to chemical conversion, separation, and/or crystallization of cannabigerolic acid (CBGA) and derivatives thereof using supercritical fluid without an extraction step, such as when CBGA and/or derivatives thereof are pre-extracted or are formulated by another method. Methods of decarboxylating cannabinoids are known (see U.S. Patent Publication Serial No. 2012/0046352 to Hospodor), the contents of which is incorporated by reference herein in its entirety. Methods of crystallization are known (U.S. Pat. No. 5,360,478 Krukonis et al. (Pando et al. 2016), the contents of which is incorporated by reference herein in its entirety.

The CBGA-containing plant material for extraction may include cannabis, particularly, for example, cannabis flowers and/or leaves, which may have a higher cannabinoid than other parts of the CBGA-containing plant material. However, the method of the present description may be employed with any part of the CBGA-containing plant material. The cannabis may preferably be industrial hemp containing no more than 0.3% THC by dry weight. However, the method may be employed for cannabis containing higher amounts of THC. The CBGA-containing plant material preferably includes a high amount of CBGA. For example, the CBGA-containing plant material preferably includes at least 2% CBGA by dry weight, preferably at least 5% CBGA by dry weight, more preferably at least 8% CBGA by dry weight.

The step of preparing the CBGA-containing plant material preferably includes grinding the CBGA-containing plant material into a powder, which facilitates further drying of the CBGA-containing plant material and facilitates an efficiency of extracting components of the CBGA-containing plant material during an extraction process. For example, industrial hemp flowers containing a high amount of CBGA may be ground into a powder having an average size of 1000 um or less, preferably 500 um or less, more preferably 200 um or less.

The step of preparing the CBGA-containing plant material preferably includes drying the CBGA-containing plant material, which facilitates efficiency of extracting components of the CBGA-containing plant material during an extraction process. The drying may continue until achieving a moisture level of less than 10% by weight, more preferably less than 5% by weight, even more preferably less than 3% by weight. Drying may be conducted by, for example, segmenting ground CBGA-containing plant material (e.g. hemp flowers containing a high amount of CBGA) into sacks for drying.

The dried, ground CBGA-containing plant material may be placed into the extractor (1). For example, the dried, ground CBGA-containing plant material may be placed into a holding container, and the holding container may be placed into the extractor (1).

The amount of the dried, ground CBGA-containing plant material placed into the holding container may be measured, e.g. weighed. Preferably, a predetermined amount (e.g. weight) of the dried, ground CBGA-containing plant material is placed into the holding container.

The holding container containing the dried, ground CBGA-containing plant material may be placed into the extractor (1) by, for example, lowering the holding container into an opening at the top of the extractor (1) and then sealing the opening at the top of the extractor (1).

The method of the present description may include a step of exposing the CBGA-containing plant material to the carbon dioxide in a supercritical state to extract CBGA from the CBGA-containing plant material. By exposing the CBGA-containing plant material to the carbon dioxide in a supercritical state, CBGA and other components of the CBGA-containing plant material are extracted from the CBGA-containing plant material. Referring to FIG. 1, the step of exposing the CBGA-containing plant material to the carbon dioxide in a supercritical state may occur within the extractor (1).

The step of exposing the CBGA-containing plant material to the carbon dioxide in a supercritical state may include exposing the CBGA-containing plant material to the carbon dioxide in a subcritical state and then transforming the carbon dioxide from the subcritical state to the supercritical state. This may include providing carbon dioxide in a subcritical liquid state to the extractor (1) containing the CBGA-containing plant material and then transforming the carbon dioxide within the extractor (1) from the subcritical state to a supercritical state.

The method of the present description preferably includes crystallizing CBGA within the carbon dioxide. In one aspect of the present description, CBGA crystals are nucleated within the carbon dioxide while in the first separator (2). In this case, the conditions (e.g. temperature, pressure, CO2 flow rate, solubility, presence of enzyme, etc.) within the first separator (2) determine or affect the identity (e.g., percentage of CBGA, ratio of CBGA and CBGA derivatives, etc.) of crystals coalesced within the first separator (2).

In another preferred aspect of the present description, CBGA crystals are nucleated within the carbon dioxide while in the extractor (1) rather than being nucleated only with the first separator (2). By nucleating CBGA crystals with the extractor (1), the conditions (e.g. temperature, pressure, CO2 flow rate, solubility, presence of enzyme, etc.) within the extractor may determine or affect the identity (e.g., percentage of CBGA, ratio of CBGA and CBGA derivatives, etc.) of crystals coalesced within the first separator (2). It is believed that crystallizing CBGA within the supercritical fluid within the extractor (1) has enabled for production of higher purity CBGA crystals coalesced in the first separator (2) as well as for production of desired ratios of CBGA and CBGA derivatives with the crystals coalesced in the first separator (2). The fact of CBGA crystallization within the supercritical fluid is confirmable, for example, by the presence of crystals of CBGA formed within the extractor (1) and deposited within the extractor (1), such as on the holding container holding the CBGA-containing plant material.

The present description includes any method of facilitating nucleation of CBGA crystals within the carbon dioxide within the extractor (1). Nucleation of crystals of CBGA within the carbon dioxide within the extractor (1) can be facilitated by controlling conditions (e.g. temperature, pressure, CO2 flow rate, solubility, presence of enzyme, etc.) within the extractor (1). For example, nucleation of CBGA crystals within the carbon dioxide within the extractor (1) can be facilitated at the initial stages of the process when the carbon dioxide is transformed from the subcritical state to the supercritical state.

Transforming the carbon dioxide from the subcritical state to the supercritical state may include increasing the temperature and/or pressure of the carbon dioxide from subcritical conditions to supercritical conditions. Carbon dioxide has a supercritical pressure of about 73.8 bars of pressure has a supercritical temperature of about 31.1° C. Thus, carbon dioxide in the subcritical state may be transformed to the supercritical state by passing from subcritical pressure to supercritical pressure, by passing from subcritical temperature to supercritical pressure, or by passing from subcritical pressure and temperature to supercritical pressure and temperature.

The pressure of the carbon dioxide within the extractor may be increased from below 73.8 Bars of pressure to above 73.8 Bars of pressure. By way of typical example, liquid carbon dioxide may be initially filled in the extractor (1) to reach 35 to 45 Bars of pressure, and high pressure pump (6) may flow carbon dioxide into the extractor (1) under higher pressures to increase the pressure of the carbon dioxide to exceed a supercritical pressure of above 73.8 Bars of pressure. The pressure build may be enabled whereby the high-pressure pump (6) is set to a pressure gradient of, for example, 7 kg/min. Note that further increasing the pressure of the carbon dioxide above supercritical pressure of above 73.8 Bars increases solubility of the CBGA within the supercritical carbon dioxide, thus increasing an efficiency of the process, but is also believed to affect chemical conversion of CBGA into CBGA derivatives as will be further explained.

The temperature of the carbon dioxide within the extractor may be increased from below 31.1° C. to above 31.1° C. by way of a heater. For example, the temperature may be increased from a range of 20 to 30° C. to a range of 31.2 to 55° C. by heating the carbon dioxide within the extractor (1). Note that further increasing the temperature of the carbon dioxide within the extractor above 31.1° C. may increase the efficiency of the process, but is also believed to affect chemical conversion of CBGA into CBGA derivatives as will be further explained.

Transforming the carbon dioxide from the subcritical state to the supercritical state may include increasing both the temperature and pressure of the carbon dioxide from subcritical temperature and pressure to supercritical temperature and pressure. Thus, for example, while the pressure is increased within a range of 35-73.8 bar, the temperature may be increased within a range of 20 to 31.1° C. Likewise, for example, while the pressure increased in a range of 73.8 bar to 300 bar, the temperature may be increased in range of 31.1° C. to 55° C.

Nucleation of crystals of CBGA within the carbon dioxide within the extractor (1) have been facilitated by controlling conditions within the extractor (1), particularly by applying a temperature gradient to the carbon dioxide within the extractor (1) during the transition from the subcritical state to the supercritical state, rather than holding the temperature of carbon dioxide within the extractor (1) to be constant during the transition from the subcritical state to the supercritical state. The temperature gradient facilitated a nucleation of CBGA crystals within the extractor (1) while approaching or passing through supercritical conditions. In particular, it is believed that the temperature gradient facilitated the formation of first isolates of CBGA in the subcritical carbon dioxide, of which growth and continued formation of the CBGA crystals in the extractor (1) was sustained after reaching supercritical conditions.

Thus, in a preferred aspect of the present description, the temperature of the carbon dioxide within the extractor (1) is not held to constant supercritical temperature while the pressure is increased from the subcritical pressure to the supercritical pressure. Rather, the temperature of the carbon dioxide is increased while the pressure is increased from a subcritical pressure to a supercritical pressure. This temperature increase may include increasing the temperature before passing from subcritical pressure to supercritical pressure. The temperature increase may include increasing the temperature while passing from subcritical pressure to supercritical pressure. The temperature increase may include increasing the temperature after passing from subcritical pressure to supercritical pressure. The necessary amount of the temperature increase is undetermined. The amount of the temperature increase, i.e. temperature gradient, is preferably at least 1° C., more preferably at least 2° C., more preferably at least 3° C., more preferably at least 5° C., more preferably at least 7° C., more preferably at least 10° C. The temperature increase has included increasing the temperature from a subcritical temperature to a supercritical temperature to facilitate nucleation of crystals of CBGA within the carbon dioxide within the extractor (1). However, the temperature increase may include increasing the temperature from a lower supercritical temperature to a higher supercritical temperature while the pressure is increased from a subcritical pressure to a supercritical pressure.

Although the invention of the present description is not limited by theory, it is believed that applying the temperature gradient to the carbon dioxide within the extractor (1), rather than holding the temperature of carbon dioxide within the extractor (1) to be constant, has facilitated the nucleation of CBGA crystallization within the extractor (1) while approaching or passing through supercritical conditions. In particular, it is believed that the temperature gradient has facilitated the formation of first isolates of CBGA in the subcritical carbon dioxide, of which growth and continued formation of the CBGA crystals in the extractor (1) is sustained after reaching supercritical conditions. The fact of CBGA crystallization within the supercritical fluid with the extractor (1) has been confirmed by the presence of crystals of CBGA formed within the extractor (1) and deposited within the extractor (1) on a holding container holding the CBGA-containing plant material. The present description includes any method of facilitating the nucleation of CBGA crystals within the carbon dioxide within the extractor (1), and, in particular, by application of a temperature gradient while the pressure is increased from a subcritical pressure to a supercritical pressure.

The method of the present description includes flowing the carbon dioxide in the supercritical state through the extractor (1) to the first separator (2). This may include opening a valve (9) between the extractor (1) and the first separator (2). The valve (9) may be opened, for example after the supercritical conditions are reached. In an aspect, the value may be opened after the temperature gradient is applied. In another aspect, the value may be opened while the temperature gradient is applied such that the temperature is continued to be increased after the valve (9) is opened. In other words, the increase in temperature within the extractor (1) may occur in part or in whole before the valve (9) between the extractor (1) and the first extractor (2) is opened to start the separation process. Thus, the effect of the temperature gradient applied within the extractor (1) on nucleation of CBGA crystals may occur prior to flowing carbon dioxide from the extractor (1) to the first separator (2).

After opening the valve (9), the high pressure pump (6) may continue to operate to flow carbon dioxide in the supercritical state through the extractor (1) to the first separator (2). The amount of carbon dioxide flowing the system may be measured, such as by way of the rotameter (5). For example, the flow rate of supercritical carbon dioxide may be fixed as in kg/min of supercritical carbon dioxide. In an aspect, the flow rate may increase over time such a flow rate gradient is applied. By starting with a low flow rate and increasing the flow rate over time, the nucleation of CBGA crystals may be promoted while at the low flow rate, and cannabinoid concentration ratio may be affected over time, facilitating for time-based separation of the crystals in the first separator (2).

The method of the present description includes coalescing a fraction of the components extracted from the CBGA-containing plant material in the extractor (1). By way of the term “fraction”, the method of the present description separates, by way of the first separator (2) the components extracted from the CBGA-containing plant material into at least one heavy fraction that coalesces and at least one light fraction that does not coalesce. The conditions within the first separator (2) determine the contents of the heavy fraction and the light fraction. Typically, the light fraction may be flowed out of the first separator. Alternatively, the light fraction could be coalesced within the first separator (2) at a later point in time. The coalesced fraction of the extracted components includes cannabinoid-containing crystals containing at least one of CBGA and a cannabinoid derivative of CBGA from the carbon dioxide.

The coalescing may be effectuated in any known manner. For example, the coalescing of crystals in the first separator (2) may be facilitated by decreasing a solubility of the carbon dioxide. In an example, the pressure of the carbon dioxide within the first separator (2) may be lower than the pressure of the carbon dioxide within the extractor (1). Thus, when carbon dioxide enters the first separator (2), a driving force exists for coalescing of crystals within the first separator (2). In a preferred example, the pressure of the carbon dioxide within the first separator (2) may be maintained above the critical pressure of 73.8 Bars of pressure. Alternatively, the pressure of the carbon dioxide within the first separator (2) may be maintained below the critical pressure of 73.8 Bars of pressure.

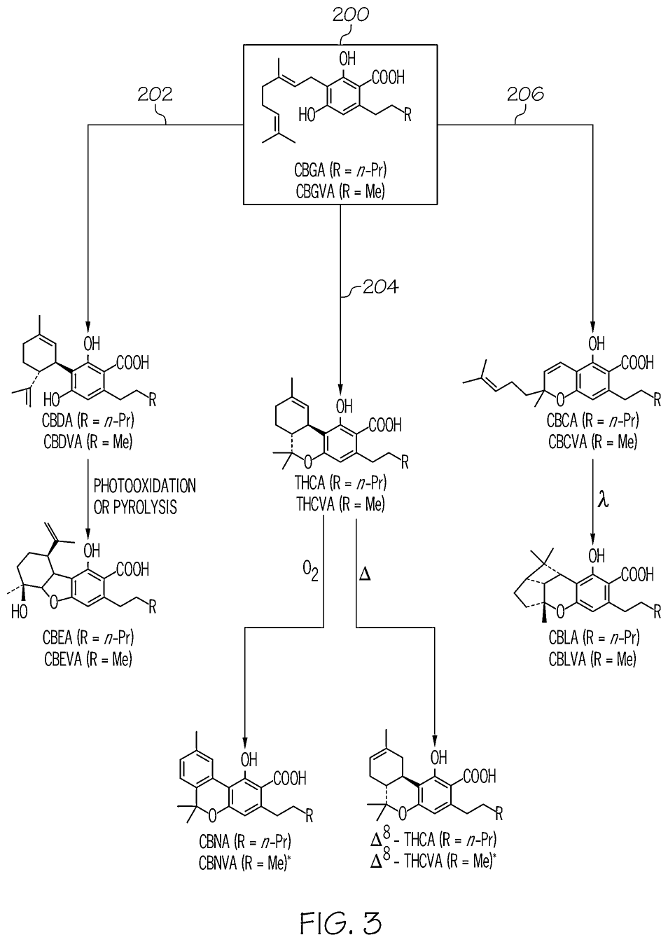

In an aspect, the crystals coalesced in the first separator (2) include crystals containing at least one of CBGA and a cannabinoid derivative of CBGA. FIG. 3 shows a non-exhaustive list of CBGA and cannabinoid derivatives of CBGA, including (i) CBGA, (ii) CBDA, THCA, and CBCA derived from CBGA, and (iii) derivatives of CBDA, THCA, and CBCA. Particularly, FIG. 3 represents a molecular conversion illustration of CBGA (200) converting to CBDA (202), THCA (204), and CBCA (206) with their derivatives.

In an aspect, the crystals coalesced in the first separator (2) include crystals containing CBGA. In an aspect, the coalesced crystals include crystals containing a high concentration of CBGA. In an example, crystals coalesced from the supercritical fluid may include crystals containing at least 50%, by weight percent, CBGA. In another example, crystals coalesced from the supercritical fluid may include crystals containing at least 60%, by weight percent, CBGA. In yet another example, crystals coalesced from the supercritical fluid may include crystals containing at least 70%, by weight percent, CBGA. In yet another example, crystals coalesced from the supercritical fluid may include crystals containing at least 80%, by weight percent, CBGA. In yet another example, crystals coalesced from the supercritical fluid may include crystals containing at least 90%, by weight percent, CBGA. These high concentration of CBGA may be achieved by, for example, initiating crystallizing of CBGA within the supercritical fluid within the extractor (1), such as by way of applying a temperature gradient during the step of exposing the CBGA-containing plant material to the carbon dioxide in a supercritical state to extract CBGA from the CBGA-containing plant material. By initiating crystallizing of CBGA within the supercritical fluid within the extractor (1), the first crystals coalesced with the first separator (2) are nearly pure CBGA. The first crystal coalesced with the first separator (2) may be separated from the crystals subsequently coalesced with the first separator (2) to obtain a high percentage of CBGA.

Furthermore, the temperature applied sustained within the extractor (1) after the temperature gradient is applied may affect the amount of CBGA extraction over time

Furthermore, a temperature within the first separator may be optimized to sustain CBGA crystal formation. In an example, a temperature within the first separator may be maintained in a preferable range of 30° C. to 35.5° C. to sustain CBGA crystal formation, more preferably in a range of 31° C. to 34.5° C., more preferably in a range of 31.5° C. to 34° C., more preferably in a range of 32° C. to 33.5° C.

In another aspect, the crystals coalesced in the first separator (2) include crystals containing at least one cannabinoid derivative of CBGA. FIG. 3 shows a non-exhaustive list of cannabinoid derivatives of CBGA. The cannabinoid derivative(s) of CBGA may have been formed in CBGA-containing plant material and extracted therefrom, or the cannabinoid derivative(s) of CBGA may result chemical conversion of CBGA that is extracted from the CBGA-containing plant material.

The composition of the crystals coalesced in the first separator (2) may be varied over time. More particularly, the cannabinoid concentration ratio within the coalesced crystals may be varied over time in a reliable manner such that the varying of the cannabinoid concentration ratio over time is substantially the same for a first batch of CBGA-containing plant material, compared to a second batch of CBGA-containing plant material, compared to the third batch of CBGA-containing plant material, etc. By way of varying the cannabinoid concentration ratio over time in a reliable manner from batch to bath, it can be possible to segregate crystals coalesced in early stages of multiple batches from crystals coalesced in later stages of multiple batches.

The varying of the cannabinoid concentration ratio over time may result from multiple effects. The varying of the cannabinoid concentration ratio over time may result, in part, from inevitable changes in the extraction of components from the CBGA-containing plant material. The varying of the cannabinoid concentration ratio over time may result, in part, from the temperature gradient imposed in the extractor (1) that causes changes in the extraction of components from the CBGA-containing plant material over time as the temperature changes. The varying of the cannabinoid concentration ratio over time may result, in part, from the temperature gradient imposed in the extractor (1) that causes changes in the crystallization within the extractor (1) over time as the temperature changes. The varying of the cannabinoid concentration ratio over time may result, in part, from the chemical conversion of extracted CBGA to derivatives of CBGA. The chemical conversion may be affected by, for example, conditions with the first separator (2), such as temperature, pressure, solubility, presence of enzymes, etc. with the first separator (2). By recognizing that the cannabinoid concentration ratio can vary over time in a reliable manner from batch to batch, it is possible to collect crystals coalesced in the first separator (2) based on time to capture a target cannabinoid concentration ratio.

Furthermore, the varying of the cannabinoid concentration ratio over time may be intentionally controlled by changing the conditions (e.g. temperature and/or pressure) of the first separator (2). For example, by increasing or decreasing a temperature of the first separator (2), the cannabinoid concentration ratio of the crystals coalesced in the first separator (2) can be controlled. It is believed that increasing or decreasing a pressure of the first separator (2) will likely have a similar effect of controlling the cannabinoid concentration ratio of the crystals coalesced in the first separator (2). By recognizing the effect of the conditions of the first separator (2) on the cannabinoid concentration ratio, it is possible to target for collection of crystals coalesced in the first separator (2) based on a target cannabinoid concentration ratio.

The varying of the cannabinoid concentration ratio based on the conditions (e.g. temperature, pressure, CO2 flow rate, solubility, presence of enzyme, etc.) of the extractor (1) may involve chemical conversion of extracted CBGA to derivatives of CBGA. Likewise, the varying of the cannabinoid concentration ratio based on the conditions (e.g. temperature, pressure, CO2 flow rate, solubility, presence of enzyme, etc.) of the first separator (2) may involve chemical conversion of extracted CBGA to derivatives of CBGA. More particularly, it has been found that temperature (e.g. temperature gradient or sustained temperature after temperature gradient) applied within the extractor (1) may affect chemical conversion of CBGA that is extracted from the CBGA-containing plant material, and that temperature applied within the first separator (2) affects chemical conversion of the CBGA.

Further, the chemical conversion of CBGA may vary over time in a reliable manner from a first extraction process to a subsequent extraction process. By varying the chemical conversion of the CBGA over time, a ratio of the cannabinoids (cannabinoid concentration ratio) of coalesced crystals varies over time in a reliable manner. By varying the cannabinoid concentration ratio of coalesced crystals over time in a reliable manner, this allows for separation of CBGA and CBGA derivatives by way of a time-based collection technique, enabling for production of high purity of CBGA crystals and for production of desired ratios of CBGA and CBGA derivatives.

In an aspect, the method of the present description may be optimized to avoid chemical conversion to maximize the concentration of CBGA. For example, the temperature of the first separator (2) may be maintained at a determined temperature to manage a desired CBGA ratio creation and said derivative cannabinoids ratios. In an example, a temperature within the first separator may be maintained in a preferable range of 30° C. to 35.5° C. to sustain CBGA crystal formation, more preferably in a range of 31° C. to 34.5° C., more preferably in a range of 31.5° C. to 34° C., more preferably in a range of 32° C. to 33.5° C.

Additionally, the ability to control the cannabinoid concentration ratio using conditions of the extractor (1) and the first separator (2) may be combined with timed collection to further manage the maximization of CBGA content and/or the collected cannabinoid concentration ratio of the coalesced crystals. In particular, crystals coalesced in the first separator (2) may be collected at different times based on desired ratios of CBGA and derivative cannabinoids and combined with collected crystals from other batches having the same or similar ratios produced under similar temperature and pressure conditions. In this regard, coalesced crystals may be continuously collected and then segmented based on time and conditions.

The method of the present description may further include flowing the carbon dioxide through the first separator (2) to the second separator (3). This may include opening a valve (9) between the first separator (2) and the second separator (3). The high pressure pump (6) may continue to operate to flow carbon dioxide in the supercritical state through the extractor (1) to the first separator (2) to the second separator (3).

The method of the present description may further include coalescing crystals from the supercritical fluid in the second separator (3). The coalescing may be effectuated in any known manner. For example, the coalescing of crystals in the second separator (3) may be facilitated by decreasing a solubility of the carbon dioxide within the second separator (3). In an example, the pressure of the carbon dioxide within the second separator (3) may be lower than the pressure of the carbon dioxide within the first separator (2). Thus, when carbon dioxide enters the second separator (3), a driving force exists for coalescing of crystals within the second separator (3).

If the amount of the pressure drop within the second separator (3) is modest, the second separator (3) may be effectuated to selectively coalesce cannabinoid-containing crystals, in which the temperature of the second separator (3) may be varied to vary a cannabinoid concentration ratio of the coalesces crystals. The coalesced crystals include crystals containing at least one of CBGA and a cannabinoid derivative of CBGA.

In an example, the pressure of the carbon dioxide within the first separator (2) may be maintained above the critical pressure of 73.8 Bars of pressure. In another example, the pressure of the carbon dioxide within the first separator (2) may be maintained below the critical pressure of 73.8 Bars of pressure.

If the amount of the pressure drop within the second separator (3) is high, then the second separator (3) may be employed to coalesce substantially all remaining cannabinoids extracted from the CBGA-containing plant material in the extractor (1) and passed through the first extractor (2) and second extractor (3). The coalesced crystals include crystals containing at least one of CBGA and a cannabinoid derivative of CBGA. For example, the pressure of the carbon dioxide within the first separator (2) may be decreased well below the critical pressure of 73.8 Bars of pressure to coalesce substantially all remaining cannabinoids.

If the second separator (3) is effectuated to selectively coalesce cannabinoid-containing crystals, then a third separator (not illustrated) may be employed. The third separator may selectively coalesce cannabinoid-containing crystals or may coalesce substantially all remaining cannabinoids. Additional separators may be included.

At the end of the extraction process, the CBGA-containing plant material may be removed from the extractor (1) and replaced with a fresh CBGA-containing plant material. Total run time for a single batch may be calculated based on total carbon dioxide flow, container volume and weight of powder of CBGA-containing plant material placed in the extractor (1).

The coalesced crystals from the first separator (2), the second separator (3), and any additional separators may be post-processed with a wash of the output material from the separation vessels. The wash may include any process for separating the cannabinoid-containing crystals from undesirable substances. For example, the wash may include dissolving the output material containing the cannabinoid-containing crystals in a solvent (e.g. ethanol), separating undesirable substances (e.g. waxes) from the solvent, and then removing the solvent (e.g. by reducing pressure). An exemplary, non-limiting, washing protocol may include:

1. Crude CO2 extracted fraction is mixed with food-grade Ethanol 200 Proof.

2. Ethanol ratio mixed with extracted crude in a range of 15:1 to 20:1.

3. Extracted fraction Wax removal process: (a) The mixture is placed in a Rotary evaporator (rotovap) 20 L rotovap evaporative flask (or Ecodyst); (b) The rotovap mixture is warmed to 50c in a water bath; (c) Vacuum on rotovap is set to 120-180 mbar; (d) Run rotovap under the above conditions till Ethanol is no longer presence; (e) Cooling down 40 RPM no vac no temp; (f) Allow solid material in rotovap to come back to room temp.

4. Activated Carbon Scrub: (a) The mixture is passed through an activated carbon column; (b) Repeat carbon scrub—Activating the carbon and wash with Ethanol; (c) Carbon scrub mixture—0.45 um filtration.

5. Initial Wash Phase: (a) Pentane is added to the rotovap under 1 atm˜900 mbar at 40c; (b) Unwashed crystal residual solvent is evaporated off; (c) Pentane is used to dissolve crystals off side of the glass flask; (d) The mixture is transferred to a container; (e) mechanically mixed to break up any larger collected crystals; (f) The mixture is smeared on the side of the container to achieve a greater surface area and better separation from the solvent.

6. Crystal seeds (CBGA and or CBDA, CBG, CBCA, CBC ETC): 10 grams—of CBGA seed crystals are added to the mixture (most parts will crystalize); (b) 20 L rotovap evaporative flask is weighed without mixture; (c) After removal of Pentane and compared to material weight in flask remaining; (d) Material is weighed and Total mass total volume is calculated.

7. Quick crystallization: (a) Disperse containers and equals and previous runs seed the matrix; (b) Container with the mixture is held at room temperature; (c) The container is then placed in −20c for 24 hours; (d) The smaller reactor could shorten the duration or full crystallization; (e) The solution is allowed to settle forming more pure crystals out of solution.

8. Filter Solution: (a) The mixture of Pentane and crystals run through Büchner funnel filter; (b) Filter used is a Whatman 1 um-0.45 um; (c) Fresh Pentane wash on the highly concentrated mixture “mother liquor”; (d) THCA washes out and remains; (e) Remaining Residual chlorophyll is washed out; (f) White dense power is mechanically pulled from the Büchner filter flask.

9. Crystal Drying: (a) Crystals are broken up into a powder and distributed in pans; (b) Pans are placed in Vacuum oven without heat; (c) Vacuum at 3.5 mbar; (d) 24 hr vacuum drying for the final stage.

The methods of the present description enable for achievement of solvent-free crystallization creation of CBGA without a high-pressure expansion and or requirement of a nozzle to achieve high expansion required in other disclosures. The driving force for nucleation and growth of the solute is supersaturation. However, in the case of the present description, crystallization is achieved in the presence of a temperature gradient that would sustain crystal production within SCCO₂ extraction and collection. Furthermore, the method of time based collection of material through the processing enables a control of cannabinoids concentration ratios in each batch collection.

The methods of the present description enable for providing an extract to create in situ isolate, containing CBGA from hemp plant material, whereby dry plant matter is extracted with CO₂ under conditions of supercritical pressure and temperature, at a temperature within the limits from 31 to 50° C. and a pressure within the limits from 75 to 300 bar. Within subcritical temperature limits from 20 to 30° C. and sub to supercritical pressure limits from 35-75 bar; and the prompting of the first isolate separated under approaching supercritical conditions.

The present specification relates to the separation and in situ refinement of cannabigerolic acid (CBGA) using Supercritical CO₂. More specifically, this invention relates to methods of controlling and influencing crystal creation of kilograms of CBGA quantities in supercritical CO₂, suitable for pharmaceutical and nutraceutical applications. The process also greatly reduces the complicated and numerous solvent-based steps required in other methods to achieve such quality and concentration while maintaining the acid to sustain CGBA efficacy throughout the process.

The present specification discloses methods of obtaining and purifying CBGA crystals from plant material as well as subsequent processing of the extract to enable for providing a 99%+ concentrate of CBGA microcrystals utilizing supercritical CO₂. The application of an initial temperature gradient just prior to supercritical state and sustained at a constant temperature through the supercritical state while maintaining target pressure initiates the forming isolate prior to sublimation in a cascading supercritical CO₂ separation. The present invention also provides for CBGA, which is the precursor of other types of cannabinoids, and the direct precursor of tetrahydrocannabinolic acid (THCA), cannabidiolic acid (CBDA), and cannabichromenic acid (CBCA). The invention relates to a method for extraction and isolated or purified cannabigerolic acid (CBGA) from industrial hemp, designed for medicinal purposes, and also the preparation of an extract within an initial temperature gradient.

It is unknown till now if any prior art process has created kilograms of CBGA isolate crystals and the mechanism of accelerated nucleation in a single process of extraction. The initial time-based temperature and pressure gradient approach is significant in the formulation of crystals within supercritical carbon dioxide. The present specification fulfills an unmet need by providing concentrated preparations of purified CBGA that do not contain solvent, and that were not prepared using any solvent to achieve ultra-concentrations of CBGA in a single closed process.

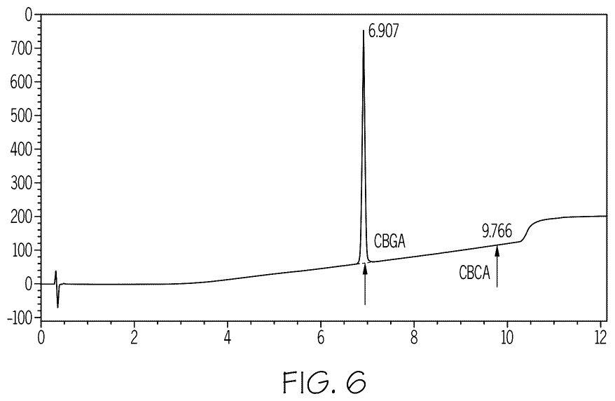

FIG. 6 shows a CBGA isolate according to the present description as measured on high-performance liquid chromatography. As shown in FIG. 6, the resulting isolate was substantially pure CBGA, i.e. at least 90%, by weight percent, CBGA. By minimizing chemical conversion of the CBGA, coalesced cannabinoid-containing crystals can be collected that include cannabinoid-containing crystals containing at least 90%, by weight percent, CBGA.

The present specification discloses methods for time based temperature gradient control crystallization to create CBGA isolate and/or a ratio of directive cannabinoids collected on a time segment. It is unknown till now if any prior art process enabled for these effects.

The conversion of CBGA into other cannabinoids such as CBDA, THCA, and CBCA using biosynthesis was known, however, controlling and influencing CBGA in a supercritical carbon dioxide process to produce CBDA, CBCA and THCA in quantity have yet to be disclosed. The transition to supercritical through the temperature and pressure gradient using a programmatic software controller created an ideal method management system for crystal isolation in the SCCO₂ apparatus. The temperature gradient method alters the solubility levels through a pressure density phase enabling the coalescence of other cannabinoids and more particularly CBGA. By holding the temperature just above supercritical the process would initiate and continue to achieve large quantities of CBGA.

The following experimental examples were conducted under the following conditions.

An industrial hemp that included a high concentration of CBGA was selected as a starting cannabis plant material. A flower sample of a typical industrial hemp that has been used for extraction was tested by high-performance liquid chromotography showing the presence of specific cannabinoids in acid and non-acid forms. Table 1 below shows the measured results in percentage (%) and in (mg/g) of cannabinoids present in the tested flower sample.

TABLE 1

| | Result | Result |
|---|---|---|
| Cannabinoid | (%) | (mg/g) |
| CBDV | N/D | N/D |
| CBDVA | N/D | N/D |
| THCV | N/D | N/D |
| CBD | N/D | N/D |
| CBG | 0.22% | 2.16 |
| CBDA | N/D | N/D |
| CBGA | 12.89% | 128.91 |
| CBN | N/D | N/D |
| THCD9 | N/D | N/D |
| THCD8 | N/D | N/D |
| CBC | N/D | N/D |
| CBNA | N/D | N/D |
| THCA | 0.11% | 1.08 |
| CBCA | 0.43% | 4.34 |

The starting cannabis plant material was ground into a powder having an average size of 1000 um or less to facilitate drying. Next, the ground cannabis plant material was dried until achieving a moisture level of less than 10% by weight. The dried, ground cannabis plant material was placed into a holding container and the holding container was placed into an extractor.

Carbon dioxide in a subcritical liquid state was provided to the extractor containing the cannabis plant material. The carbon dioxide within the extractor was then transformed from the subcritical state to a supercritical state to extract components from the starting plant material, and then the carbon dioxide containing the extracted components of the starting plant material was flowed to a first separator. A pressure of the carbon dioxide flowed into the first separator was decreased to coalesce a fraction of the extracted components from the carbon dioxide. The coalesced fraction of the extracted components included cannabinoid-containing crystals containing CBGA and a cannabinoid derivative of CBGA. The carbon dioxide containing an uncoalesced fraction of the extracted components was flowed out of the first separator to a second separator. The cannabinoid-containing crystals were collected from the first separator.

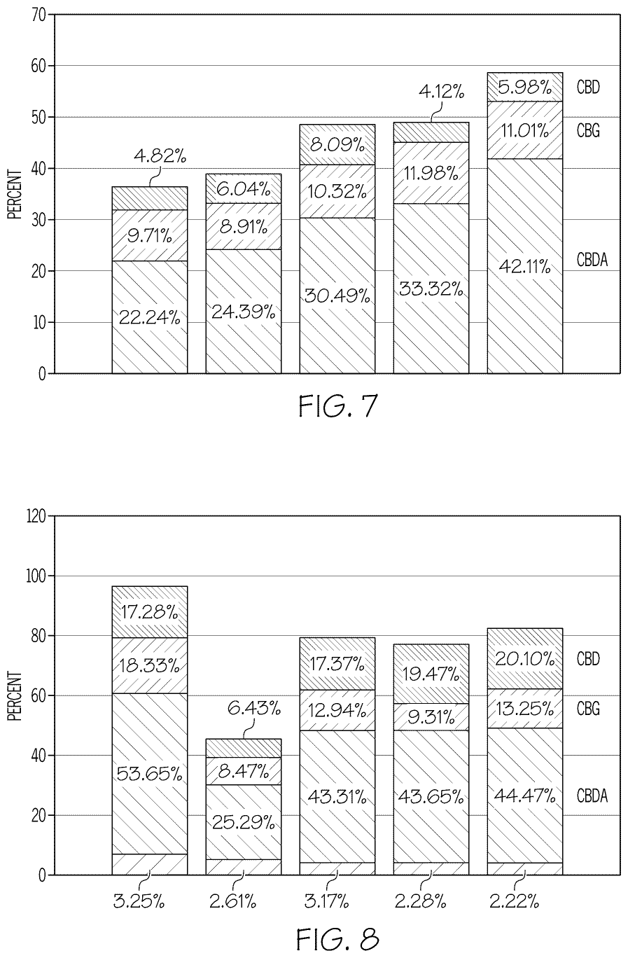

Experimental Example 1—Pressure, Temperature, and CO2 Flow Rate Held Constant. FIG. 7 is a graph representing a typical experimental result when carbon dioxide within the extractor was transformed from the subcritical state to a supercritical state and then the pressure, the temperature and CO2 flow rate were held constant as CO2 flowed from the extractor to the separator. The cannabinoid-containing crystals were collected at intervals of 20%, 40%, 60%, 80%, and 100% of the total run time. As shown by FIG. 7, an output yield of cannabinoid-containing crystals peeked at the end of the run with low overall extraction efficiency. This example also demonstrates that it is possible to collect coalesced crystals coalesced based on time to capture a target cannabinoid concentration ratio.

Experimental Example 2—Gradient Applied. FIG. 8 is a graph representing a typical experimental result when pressure and temperature are increased as a gradient over time during transformation of carbon dioxide within the extractor from the subcritical state to a supercritical state and during flow of CO2 from the extractor to the separator and when flow of CO2 was increased as a gradient during flow of CO2 from the extractor to the separator. The cannabinoid-containing crystals were collected at intervals of 20%, 40%, 60%, 80%, and 100% of the total run time. As shown by FIG. 8, the beginning of the crystallization has a clear increase in output yield of cannabinoid-containing crystals at the beginning and level out over the remainder of the run with higher overall extraction efficiency compared with Experimental Example 1.

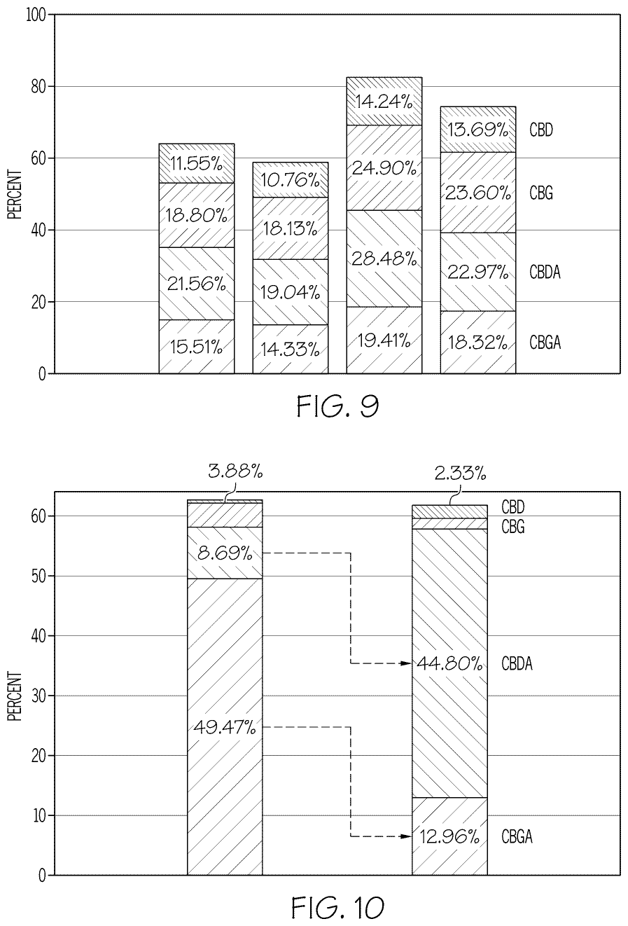

Experimental Example 3—Short Gradient vs. Long Gradient. FIG. 9 is a graph representing a typical experimental result when pressure, temperature, and flow rate are increased as a gradient over time. The left side of the graph represents two runs for which cannabinoid-containing crystals were collected at the ½ way point of the run after applying a short gradient. The right side of the graph represents two runs for which cannabinoid-containing crystals were collected at the ½ way point of the run after applying a longer gradient. As shown by FIG. 9, the runs with a longer gradient produced higher yield output on concentration of target cannabinoids.

Experimental Example 4—Control of Chemical Conversion of CBGA. FIG. 10 is a graph representing a typical experimental result when conditions within the first separator were varied to influence of the chemical conversion of CBGA. In this case, the temperature and pressure within the extractor were held constant after moving through a temperature and pressure gradient. The left side of the graph represents a run for which a temperature within the first separator was held at less than 35 degrees C., and left side of the graph represents a run for which a temperature within the first separator was held at greater than 55 degrees C. As shown by FIG. 10, the run within the lower temperature within the first separator retained a high amount of the extracted CBGA, and the run within the higher temperature within the first separator results in a high amount of chemical conversion of the extracted CBGA into cannabinoid derivatives of CBGA.

Various aspects are represented below by the following clauses. The present invention is not limited to the aspects represented in these clauses. Rather, the present invention includes these aspects in combination with any one or more additional features described above or illustrated in the encloses drawings.

Clause 1. A method for producing cannabinoid-containing crystals, the method comprising the steps of: providing a CBGA-containing plant material that comprises at least 2% CBGA by dry weight; exposing the CBGA-containing plant material to a fluid in a supercritical state to extract components of the CBGA-containing plant material; and coalescing a fraction of the extracted components, the coalesced fraction of the extracted components including cannabinoid-containing crystals containing at least one of CBGA and a cannabinoid derivative of CBGA from the carbon dioxide.

Clause 2. The method of clause 1, wherein the CBGA-containing plant material comprises cannabis.

Clause 3. The method of clause 1, wherein the CBGA-containing plant material comprises industrial hemp.

Clause 4. The method of any one of clauses 1 to 3, wherein the CBGA-containing plant material comprises at least 5% CBGA by dry weight.

Clause 5. The method of any one of clauses 1 to 4, wherein the CBGA-containing plant material comprises at least 8% CBGA by dry weight.

Clause 6. The method of any one of clauses 1 to 5, further comprising crystallizing CBGA within the fluid while in the supercritical state.

Clause 7. The method of any one of clauses 1 to 6, wherein the step of exposing the CBGA-containing plant material to the fluid in the supercritical state comprises exposing the CBGA-containing plant material to the fluid in a subcritical liquid state and then transforming the fluid to a supercritical state.

Clause 8. The method of clause 7, wherein transforming the fluid to a supercritical state comprises increasing the pressure of the fluid.

Clause 9. The method of any one of clauses 7 to 8, wherein transforming the fluid to a supercritical state comprises increasing the temperature of the fluid.

Clause 10. The method of any one of clauses 7 to 9, further comprising increasing a temperature of the fluid over time while the fluid is in the subcritical liquid state.

Clause 11. The method of any one of clauses 7 to 10, further comprising increasing a temperature of the fluid over time while the fluid transforms to the supercritical state.

Clause 12. The method of any one of clauses 7 to 11, further comprising increasing a temperature of the fluid over time while the fluid is in the supercritical state.

Clause 13. The method of any one of clauses 1 to 12, wherein the step of coalescing a fraction of the extracted components comprises reducing a pressure of the fluid.

Clause 14. The method of any one of clauses 1 to 13, wherein the step of coalescing a fraction of the extracted components comprises transforming the fluid from the supercritical state to a subcritical state.

Clause 15. The method of any one of clauses 1 to 14, wherein coalesced cannabinoid-containing crystals include cannabinoid-containing crystals containing at least 50%, by weight percent, CBGA.

Clause 16. The method of any one of clauses 1 to 15, wherein coalesced cannabinoid-containing crystals include cannabinoid-containing crystals containing at least 60%, by weight percent, CBGA.

Clause 17. The method of any one of clauses 1 to 16, wherein coalesced cannabinoid-containing crystals include cannabinoid-containing crystals containing at least 70%, by weight percent, CBGA.

Clause 18. The method of any one of clauses 1 to 17, wherein coalesced cannabinoid-containing crystals include cannabinoid-containing crystals containing at least 80%, by weight percent, CBGA.

Clause 19. The method of any one of clauses 1 to 18, wherein coalesced cannabinoid-containing crystals include cannabinoid-containing crystals containing at least 90%, by weight percent, CBGA.

Clause 20. The method of any one of clauses 1 to 19, further comprising chemically converting CBGA extracted from the CBGA-containing plant material to one or more cannabinoid derivatives of CBGA, and wherein the coalesced cannabinoid-containing crystals contain the one or more cannabinoid derivatives converted from the CBGA extracted from the CBGA-containing plant material.

Clause 21. The method of clause 20, wherein chemically converting the extracted CBGA from the CBGA-containing plant material to one or more cannabinoid derivatives of CBGA includes reacting the extracted CBGA with a cannabinoid synthase in the fluid.

Clause 22. The method of any one of clauses 1 to 21, wherein the cannabinoid-containing crystals coalesced from the fluid at a first time have a first cannabinoid concentration ratio and the cannabinoid-containing crystals coalesced from the fluid at a second time, which is later than the first time, have a second cannabinoid concentration ratio, which is different from the first cannabinoid concentration ratio.

Clause 23. The method of any clause 22, wherein first cannabinoid concentration ratio has a higher concentration of CBGA than the second cannabinoid concentration ratio.

Clause 24. The method of any one of clauses 22 to 23, further comprising segregating the cannabinoid-containing crystals coalesced at the first time from the cannabinoid-containing crystals coalesced at the second time.

Clause 25. A method for producing cannabinoid-containing crystals, the method comprising the steps of: placing CBGA-containing plant material that comprises at least 2% CBGA by dry weight into an extractor; providing carbon dioxide in a subcritical liquid state to the extractor containing the CBGA-containing plant material; transforming the carbon dioxide within the extractor from the subcritical state to a supercritical state to extract components from the CBGA-containing plant material; flowing the carbon dioxide containing the extracted components of the CBGA-containing plant material to a first separator; decreasing a pressure of the carbon dioxide flowed into the first separator to coalesce a fraction of the extracted components from the carbon dioxide, the coalesced fraction of the extracted components including cannabinoid-containing crystals containing at least one of CBGA and a cannabinoid derivative of CBGA; flowing the carbon dioxide containing an uncoalesced fraction of the extracted components out of the first separator; and collecting the cannabinoid-containing crystals from the first separator.

Clause 26. The method of clause 25, wherein the CBGA-containing plant material comprises cannabis.

Clause 27. The method of clause 25, wherein the CBGA-containing plant material comprises industrial hemp.

Clause 28. The method of any one of clauses 25 to 27, wherein the CBGA-containing plant material comprises at least 5% CBGA by dry weight.

Clause 29. The method of any one of clauses 25 to 28, wherein the CBGA-containing plant material comprises at least 8% CBGA by dry weight.

Clause 30. The method of any one of clauses 25 to 29, wherein the step of transforming the carbon dioxide within the extractor from the subcritical state to a supercritical state includes increasing a pressure of the carbon dioxide within the extractor.

Clause 31. The method of any one of clauses 25 to 30, wherein the step of transforming the carbon dioxide within the extractor from the subcritical state to a supercritical state includes increasing a temperature of the carbon dioxide over time.

Clause 32. The method of any one of clauses 25 to 31, wherein the step of transforming the carbon dioxide within the extractor from the subcritical state to a supercritical state includes increasing a temperature of the carbon dioxide over time while the carbon dioxide is in the subcritical liquid state.

Clause 33. The method of any one of clauses 25 to 32, wherein the step of transforming the carbon dioxide within the extractor from the subcritical state to a supercritical state includes increasing a temperature of the carbon dioxide over time while the carbon dioxide transforms to the supercritical state.

Clause 34. The method of any one of clauses 25 to 33, wherein the step of transforming the carbon dioxide within the extractor from the subcritical state to a supercritical state includes increasing a temperature of the carbon dioxide over time while the carbon dioxide is in the supercritical state.

Clause 35. The method of any one of clauses 25 to 34, wherein CBGA crystals are formed within the extractor.

Clause 36. The method of any one of clauses 25 to 35, wherein the step of decreasing a pressure of the carbon dioxide flowed into the first separator includes decreasing a temperature of the carbon dioxide flowed into the first separator.

Clause 37. The method of any one of clauses 25 to 36, wherein the step of decreasing a pressure of the carbon dioxide flowed into the first separator includes transforming the carbon dioxide flowed into the first separator from the supercritical state to a subcritical liquid state.

Clause 38. The method of any one of clauses 25 to 37, wherein coalesced cannabinoid-containing crystals collected from the first separator include cannabinoid-containing crystals containing at least 50%, by weight percent, CBGA.

Clause 39. The method of any one of clauses 25 to 38, wherein coalesced cannabinoid-containing crystals collected from the first separator include cannabinoid-containing crystals containing at least 60%, by weight percent, CBGA.

Clause 40. The method of any one of clauses 25 to 39, wherein coalesced cannabinoid-containing crystals collected from the first separator include cannabinoid-containing crystals containing at least 70%, by weight percent, CBGA.

Clause 41. The method of any one of clauses 25 to 40, wherein coalesced cannabinoid-containing crystals collected from the first separator include cannabinoid-containing crystals containing at least 80%, by weight percent, CBGA.

Clause 42. The method of any one of clauses 25 to 41, wherein coalesced cannabinoid-containing crystals collected from the first separator include cannabinoid-containing crystals containing at least 90%, by weight percent, CBGA.

Clause 43. The method of any one of clauses 25 to 42, further comprising chemically converting CBGA extracted from the CBGA-containing plant material to one or more cannabinoid derivatives of CBGA, and wherein the coalesced cannabinoid-containing crystals contain the one or more cannabinoid derivatives converted from the CBGA extracted from the CBGA-containing plant material.

Clause 44. The method of any one of clauses 25 to 43, wherein cannabinoid-containing crystals collected from the first separator at a first time have a first cannabinoid concentration ratio and cannabinoid-containing crystals collected from the first separator at a second time, which is later than the first time, have a second cannabinoid concentration ratio, which is different from the first cannabinoid concentration ratio.

Clause 45. The method of clause 44, wherein first cannabinoid concentration ratio has a higher concentration of CBGA than the second cannabinoid concentration ratio.

Clause 46. The method of any one of clauses 44 to 45, further comprising segregating the cannabinoid-containing crystals collected at the first time from the cannabinoid-containing crystals collected at the second time.

Although various embodiments of the disclosed methods for producing cannabinoid-containing crystals using supercritical fluid have been shown and described, modifications may occur to those skilled in the art upon reading the specification. The present application includes such modifications and is limited only by the scope of the claims.

What is claimed is:

1. A method for producing cannabinoid-containing crystals, the method comprising the steps of:
providing a CBGA-containing plant material that comprises at least 2% CBGA by dry weight;
exposing the CBGA-containing plant material to a fluid in a supercritical state to extract components of the CBGA-containing plant material; and
coalescing a fraction of the extracted components, the coalesced fraction of the extracted components including cannabinoid-containing crystals containing at least one of CBGA and a cannabinoid derivative of CBGA from the carbon dioxide.

2. The method of claim 1, wherein the CBGA-containing plant material comprises cannabis.

3. The method of claim 1, wherein the CBGA-containing plant material comprises industrial hemp.

4. The method of claim 1, wherein the CBGA-containing plant material comprises at least 5% CBGA by dry weight.

5. The method of claim 1, wherein the CBGA-containing plant material comprises at least 8% CBGA by dry weight.

6. The method of claim 1, further comprising crystallizing CBGA within the fluid while in the supercritical state.

7. The method of claim 1, wherein the step of exposing the CBGA-containing plant material to the fluid in the supercritical state comprises exposing the CBGA-containing plant material to the fluid in a subcritical liquid state and then transforming the fluid to a supercritical state.

8. The method of claim 7, wherein transforming the fluid to a supercritical state comprises increasing the pressure of the fluid.

9. The method of claim 7, wherein transforming the fluid to a supercritical state comprises increasing the temperature of the fluid.

10. The method of claim 7, further comprising increasing a temperature of the fluid over time while the fluid is in the subcritical liquid state.

11. The method of claim 7, further comprising increasing a temperature of the fluid over time while the fluid transforms to the supercritical state.

12. The method of claim 7, further comprising increasing a temperature of the fluid over time while the fluid is in the supercritical state.

13. The method of claim 1, wherein the step of coalescing a fraction of the extracted components comprises reducing a pressure of the fluid.

14. The method of claim 1, wherein the step of coalescing a fraction of the extracted components comprises transforming the fluid from the supercritical state to a subcritical state.

15. The method of claim 1, wherein coalesced cannabinoid-containing crystals include cannabinoid-containing crystals containing at least 50%, by weight percent, CBGA.

16. The method of claim 1, wherein coalesced cannabinoid-containing crystals include cannabinoid-containing crystals containing at least 60%, by weight percent, CBGA.

17. The method of claim 1, wherein coalesced cannabinoid-containing crystals include cannabinoid-containing crystals containing at least 70%, by weight percent, CBGA.

18. The method of claim 1, wherein coalesced cannabinoid-containing crystals include cannabinoid-containing crystals containing at least 80%, by weight percent, CBGA.

19. The method of claim 1, wherein coalesced cannabinoid-containing crystals include cannabinoid-containing crystals containing at least 90%, by weight percent, CBGA.

20. The method of claim 1, further comprising chemically converting CBGA extracted from the CBGA-containing plant material to one or more cannabinoid derivatives of CBGA, and wherein the coalesced cannabinoid-containing crystals contain the one or more cannabinoid derivatives converted from the CBGA extracted from the CBGA-containing plant material.

21. The method of claim 20, wherein chemically converting the extracted CBGA from the CBGA-containing plant material to one or more cannabinoid derivatives of CBGA includes reacting the extracted CBGA with a cannabinoid synthase in the fluid.

22. The method of claim 1, wherein the cannabinoid-containing crystals coalesced from the fluid at a first time have a first cannabinoid concentration ratio and the cannabinoid-containing crystals coalesced from the fluid at a second time, which is later than the first time, have a second cannabinoid concentration ratio, which is different from the first cannabinoid concentration ratio.

23. The method of claim 22, wherein first cannabinoid concentration ratio has a higher concentration of CBGA than the second cannabinoid concentration ratio.

24. The method of claim 22, further comprising segregating the cannabinoid-containing crystals coalesced at the first time from the cannabinoid-containing crystals coalesced at the second time.

25. A method for producing cannabinoid-containing crystals, the method comprising the steps of:
placing CBGA-containing plant material that comprises at least 2% CBGA by dry weight into an extractor;
providing carbon dioxide in a subcritical liquid state to the extractor containing the CBGA-containing plant material;
transforming the carbon dioxide within the extractor from the subcritical state to a supercritical state to extract components from the CBGA-containing plant material;
flowing the carbon dioxide containing the extracted components of the CBGA-containing plant material to a first separator;
decreasing a pressure of the carbon dioxide flowed into the first separator to coalesce a fraction of the extracted components from the carbon dioxide, the coalesced fraction of the extracted components including cannabinoid-containing crystals containing at least one of CBGA and a cannabinoid derivative of CBGA;
flowing the carbon dioxide containing an uncoalesced fraction of the extracted components out of the first separator; and
collecting the cannabinoid-containing crystals from the first separator.

26. The method of claim 25, wherein the CBGA-containing plant material comprises cannabis.

27. The method of claim 25, wherein the CBGA-containing plant material comprises industrial hemp.

28. The method of claim 25, wherein the CBGA-containing plant material comprises at least 5% CBGA by dry weight.

29. The method of claim 25, wherein the CBGA-containing plant material comprises at least 8% CBGA by dry weight.

30. The method of claim 25, wherein the step of transforming the carbon dioxide within the extractor from the subcritical state to a supercritical state includes increasing a pressure of the carbon dioxide within the extractor.

31. The method of claim 25, wherein the step of transforming the carbon dioxide within the extractor from the subcritical state to a supercritical state includes increasing a temperature of the carbon dioxide over time.

32. The method of claim 25, wherein the step of transforming the carbon dioxide within the extractor from the subcritical state to a supercritical state includes increasing a temperature of the carbon dioxide over time while the carbon dioxide is in the subcritical liquid state.

33. The method of claim 25, wherein the step of transforming the carbon dioxide within the extractor from the subcritical state to a supercritical state includes increasing a temperature of the carbon dioxide over time while the carbon dioxide transforms to the supercritical state.

34. The method of claim 25, wherein the step of transforming the carbon dioxide within the extractor from the subcritical state to a supercritical state includes increasing a temperature of the carbon dioxide over time while the carbon dioxide is in the supercritical state.

35. The method of claim 25, wherein CBGA crystals are formed within the extractor.

36. The method of claim 25, wherein the step of decreasing a pressure of the carbon dioxide flowed into the first separator includes decreasing a temperature of the carbon dioxide flowed into the first separator.

37. The method of claim 25, wherein the step of decreasing a pressure of the carbon dioxide flowed into the first separator includes transforming the carbon dioxide flowed into the first separator from the supercritical state to a subcritical liquid state.

38. The method of claim 25, wherein coalesced cannabinoid-containing crystals collected from the first separator include cannabinoid-containing crystals containing at least 50%, by weight percent, CBGA.

39. The method of claim 25, wherein coalesced cannabinoid-containing crystals collected from the first separator include cannabinoid-containing crystals containing at least 60%, by weight percent, CBGA.

40. The method of claim 25, wherein coalesced cannabinoid-containing crystals collected from the first separator include cannabinoid-containing crystals containing at least 70%, by weight percent, CBGA.

41. The method of claim 25, wherein coalesced cannabinoid-containing crystals collected from the first separator include cannabinoid-containing crystals containing at least 80%, by weight percent, CBGA.

42. The method of claim 25, wherein coalesced cannabinoid-containing crystals collected from the first separator include cannabinoid-containing crystals containing at least 90%, by weight percent, CBGA.

43. The method of claim 25, further comprising chemically converting CBGA extracted from the CBGA-containing plant material to one or more cannabinoid derivatives of CBGA, and wherein the coalesced cannabinoid-containing crystals contain the one or more cannabinoid derivatives converted from the CBGA extracted from the CBGA-containing plant material.

44. The method of claim 25, wherein cannabinoid-containing crystals collected from the first separator at a first time have a first cannabinoid concentration ratio and cannabinoid-containing crystals collected from the first separator at a second time, which is later than the first time, have a second cannabinoid concentration ratio, which is different from the first cannabinoid concentration ratio.

45. The method of claim 44, wherein first cannabinoid concentration ratio has a higher concentration of CBGA than the second cannabinoid concentration ratio.

46. The method of claim 44, further comprising segregating the cannabinoid-containing crystals collected at the first time from the cannabinoid-containing crystals collected at the second time.
